(19) United States
(12) Patent Application Publication
(10) Pub. No.: US 2016/0067435 A1
(43) Pub. Date: Mar. 10, 2016

(54) Endopharyngeal airway device and kit and method of use

(71) Applicant: Diana B. Thomas, Prospect, KY (US)

(72) Inventor: Diana B. Thomas, Prospect, KY (US)

(21) Appl. No.: 14/756,039

(22) Filed: Jul. 23, 2015

Related U.S. Application Data
(60) Provisional application No. 61/999,314, filed on Jul. 23, 2014.

(51) Int. Cl. A61M 16/04; A61M 16/00

(52) U.S. Cl. CPC A61M 16/0461; A61M 16/0078; A61M 16/0475; A61M 2205/3327

(57) ABSTRACT

A kit for ventilating a patient incapable of normal ventilation with current anesthetic techniques due to a risk for airway obstruction in surgical procedures requiring conscious sedation monitored anesthesia care (MAC). Includes a single-lumen endopharyngeal nasal tube with an eye shaped opening at its distal end and a cylindrical proximal end used as an adaptor for anesthetic applications such as positive airway pressure via Jackson-Reese modified ambu-bag or other various anesthesiology applications. Contiguous to the distal end of the nasal tube is an end-tidal CO2 monitoring port (ETCO2). On the distal end of the flexible nasal ventilation tube is an eye opening allowing air flow to the posterior pharynx. The kit further includes a modified Jackson-Reese ambu bag which allows for controlled airflow to the patient during a procedure.

### CROSS REFERENCE TO RELATED APPLICATIONS

This application claims priority from U.S. Provisional Application Ser. No. 61/999,314 filed on Jul. 23, 2014 which is incorporated herein in its entirety.

### FIELD OF THE INVENTION

The present invention pertains to an anesthetic device and kit and more particularly, to a endopharyngeal airway device and kit for the delivery of positive-pressure in patients at risk for airway obstruction in surgical procedures requiring conscious sedation/monitored anesthesia care (MAC).

### BACKGROUND OF THE INVENTION

Historically, the traditional nasal airway provided adequate assistance in relieving partial airway obstruction. However, in patients with obstructive sleep apnea (OSA) and morbid obesity, opening an airway is often not sufficient to ventilate the patient.

Increasing risks of airway obstruction secondary to rising incidence of obstructive sleep apena (OS) and morbid obesity; and increasing number of anesthesia cases being performed in non-traditional settings (dental offices, endoscopy suites, outpatient ambulatory surgical centers (ASC) using IV sedation with PROPOFOL or PRECEDEX. In patients with OSA or morbid obesity, opening and airway is often not sufficient to adequately ventilate the patient.

More than 60% of elective surgery procedures in the U.S. are performed in outpatient setting (15,000,000 cases), which is expected to reach 75% by the end of the next decade. Forty-to percent of all outpatient surgeries take place in an ASC. The above percentage is expected to rise due to better outpatient reimbursement (over 50% covered by private insurance), and overall cost savings of outpatient procedures over inpatient. Many outpatient/ASC procedures are typically performed using MAC conscious sedation forms of anesthesia. GI endoscopy (the most common outpatient procedure) is now mandated to be covered by all insurance.

Therefore, it would be highly desired to have a new and improved method, device and kit for establishing a positive pressure endopharyngeal airway without the need of providing a surgical airway.

### SUMMARY OF THE INVENTION

Providing direct oxygen and the ability to provide positive airway pressure is needed and provided by the instant endopharyngeal device (EPA). The invention is a modification of the current anesthesia airway to provide application of positive airway pressure in patients at isk for airway obstruction in surgical procedures requiring conscious sedation/monitored anesthesia care (MAC). It is recommended for MAC anesthesia with sedation in patients at risk for partially obstructed airways. It is not recommended for general anesthesia. It should be used with caution in patients taking anticoagulants, and it not recommended for patients with facial trauma including facial fractures.

One preferred embodiment includes an endopharyngeal airway device for ventilating a patient incapable of normal ventilation with current anesthetic techniques due to a risk for airway obstruction in surgical procedures requiring conscious sedation monitored anesthesia care. The device includes a single-lumen flexible endopharyngeal nasal ventilation tube including an eye shaped opening at its distal end and an adapter having a cylindrical proximal end removable coupling to a positive airway pressure device. An end-tidal CO2 monitoring port is contiguous to the distal end of the nasal tube. An eye opening on the distal end of the endopharyngeal nasal ventilation tube allowing air flow to the posterior pharynx. A positive airway pressure device such as a JACKSON REESE bag allows for controlled airflow to the patient during a procedure.

The present invention comprises a kit for ventilating a patient incapable of normal ventilation with current anesthetic techniques due to a risk for airway obstruction in surgical procedures requiring conscious sedation/monitored anesthesia care (MAC). Includes a single-lumen endopharyngeal nasal tube with an eye shaped opening at its distal end and a cylindrical proximal end used as an adaptor for anesthetic applications such as positive airway pressure via Jackson-Reese modified ambu-bag or other various anesthesiology applications. Contiguous to the distal end of the nasal tube is an end-tidal CO2 monitoring port (ETCO2). The distal end of the tube and proximal end of the end-tidal CO2 monitoring port are enclosed in the proximal end of the flexible nasal ventilation tube. The flexible nasal ventilation tube is made of a latex-free, non-toxic material. On the distal end of the flexible nasal ventilation tube is an eye opening allowing air flow to the posterior pharynx. Contiguous to the distal eye opening is an opening in the sidewall of the flexible nasal ventilation tube allowing for airflow to the posterior pharynx in the event that the distal eye is covered by body tissue. The kit further includes a modified Jackson-Reese ambu bag which allows for controlled airflow to the patient during a procedure. A medical practitioner using the endopharyngeal airway proceeds ventilating a patient by using neosynephrine nasal spray in the least obstructed nare, selecting a flexible nasal ventilation tube size by measuring from the tip of the nose to the earlobe or angle of jaw, applying lubricating gel to the flexible nasal ventilation tube, and inserting the flexible nasal ventilation tube into the nares with a gentle rotating motion. When the flexible nasal ventilation tube is in the correct position, it will extend from the distal end of the tube to the posterior of the pharynx.

In one preferred embodiment of the present invention, a kit for ventilating a patient incapable of normal ventilation with current anesthetic techniques due to a risk for airway obstruction in surgical procedures requiring conscious sedation/monitored anesthesia care (MAC). Includes a single-lumen endopharyngeal nasal tube with an eye shaped opening at its distal end and a cylindrical proximal end used as an adaptor for anesthetic applications such as positive airway pressure via Jackson-Reese modified ambu-bag or other various anesthesiology applications. The cylindrical, proximal end of the nasal tube is adapted to be connected in a fast, convenient manner to an air delivery device. Contiguous to the distal end of the nasal tube is an end-tidal CO2 monitoring port (ETCO2). The distal end of the tube and proximal end of the end-tidal CO2 monitoring port are enclosed in the proximal end of the flexible nasal ventilation tube. The flexible nasal ventilation tube is made of a latex-free, non-toxic material. On the distal end of the flexible nasal ventilation tube is an eye opening allowing air flow to the posterior pharynx. Contiguous to the distal eye opening is an opening in the sidewall of the flexible nasal ventilation tube allowing for airflow to the posterior pharynx in the event that the distal eye is covered by body tissue. The kit further includes a modified Jackson-Reese ambu bag which allows for controlled airflow to the patient during a procedure.

The ventilation rescue method includes providing a single-lumen endopharyngeal nasal tube having a cylindrical proximal end and an eye shaped opening at its distal end with an end-tidal CO2 monitoring port contiguous to the distal end of the tube both of which are enclosed by the flexible nasal ventilation tube. A medical practitioner using the endopharyngeal airway proceeds ventilating a patient by using neosynephrine nasal spray in the least obstructed nare, selecting a flexible nasal ventilation tube size by measuring from the tip of the nose to the earlobe or angle of jaw, applying lubricating gel to the flexible nasal ventilation tube, and inserting the flexible nasal ventilation tube into the nares with a gentle rotating motion. When the flexible nasal ventilation tube is in the correct position, it will extend from the distal end of the tube to the posterior of the pharynx. With the nasal tube so positioned the medical practitioner applies positive pressure to the modified Jackson-Reese ambu bag in synchronization with the inspiratory phase allowing sufficient time for exhalation through the mouth of the patient.

The tube size is selected by measuring from tip of the nose to earlobe or angle of the jaw. Lubricating gel is applied to the tube. The tube is inserted into the nare with gentle rotating motion. When the tube is in the correct position it will extend from the nare to the posterior pharynx. It does not extend down pass the tracheae.

It is an objective of the present invention to utilize a flexible tube made of latex free nontoxic material.

It is an objective of the present invention to provide an end-tidal carbon dioxide monitoring port (ETCO2).

It is an objective of the present invention to provide a distal eye to provide more efficient flow of air to posterior pharynx especially in case the opening in the distal end of the tube is partially blocked by tissue or other obstruction.

It is an objective of the present invention to provide an adaptor for anesthesia applications such as positive airway pressure via Jackson-Reese modified ambu-bag or ventilator.

It is an objective of the present invention to provide means for administering a neosynephrine nasal spray in one of the least obstructed nares.

It is an objective of the present invention to provide for an ETCO2 port for continuous monitoring of carbon dioxide.

It is another objective of the present invention top provide for a distal eye to improve airway patency.

It is an objective of the present invention to provide an adaptor for possible anesthesia applications including direct oxygen delivery to posterior pharynx; non-rebreather bay with pressure relief valve for positive airway pressure; JACKSON-REESE modified ambu-bag; and anesthesia circuit with PSV pro for spontaneous ventilation with pressure support.

The use of an eye, use of carbon dioxide port or which can be molded as part of the connection or “adapter” which uses a universal friction fit coupling on the end of a tube which cooperatively engages the air bag provides an inexpensive means for providing direct oxygen and the ability to provide positive airway pressure is needed.

Other objects, features, and advantages of the invention will be apparent with the following detailed description taken in conjunction with the accompanying drawings showing a preferred embodiment of the invention.

### BRIEF DESCRIPTION OF THE DRAWINGS

A better understanding of the present invention will be had upon reference to the following description in conjunction with the accompanying drawings in which like numerals refer to like parts throughout the views wherein:

FIG. 1 is a perspective view of the completely assembled device showing the distal end of the nasal tube and proximal end of the end-tidal CO2 monitor port enclosed by the flexible nasal ventilation tube with the modified Jackson-Reese ambu bag detached from the adaptor, illustrating how the adaptor for various anesthesiology applications attaches to the modified Jackson-Reese ambu bag with the nipple in flow communication with a sleeve valve, and an air delivery system via ventilation pump tube;

FIG. 2 is a pictorial view of the endopharyngeal airway device, modified Jackson-Reese ambu bag and ventilation pump tube before assembly;

FIG. 3 is a perspective enlarged view of the port to end tidal CO2 monitor;

FIG. 4 is a pictorial view, illustrating the endopharyngeal airway without various anesthesiology applications attached at the adaptor of the proximal end of the nasal tube;

FIG. 5 is a pictorial view of the eye opening at the distal end of the flexible nasal ventilation tube and the distal eye posterior to the pharynx contiguous with it;

FIG. 6 is a perspective view of the ventilation pump tube with its female and male fitting adaptor parts;

FIG. 7 is a perspective view of the modified Jackson-Reese ambu bag attached directly to the endopharyngeal nasal tube via its female fitting part;

FIG. 8 is a perspective view of only the ventilation pump tube connected directly to the endopharyngeal nasal tube;

FIG. 9 illustrates the step of inserting the endopharyngeal airway device into the nostril of the patient for passage to the posterior pharynx; and

FIG. 10 is a pictorial view, illustrating operative elements of still yet another ventilation kit, which is constructed in accordance with the present invention.

### DETAILED DESCRIPTION OF THE PREFERRED EMBODIMENT OF THE INVENTION

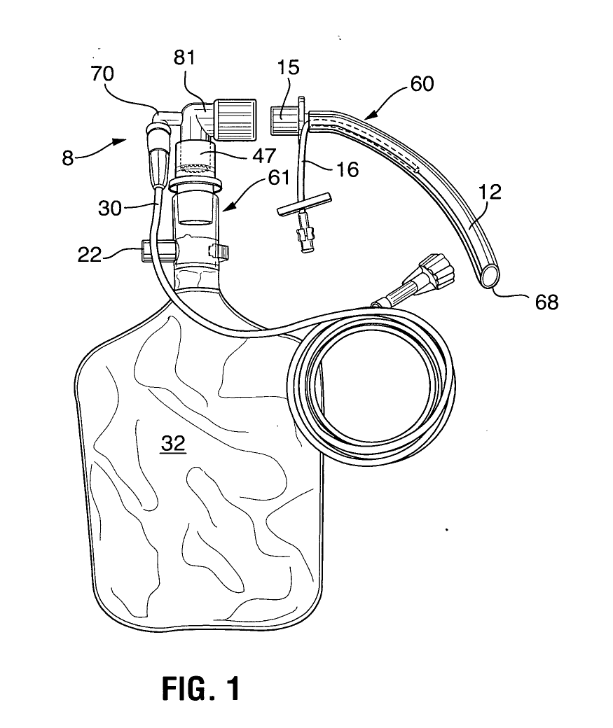

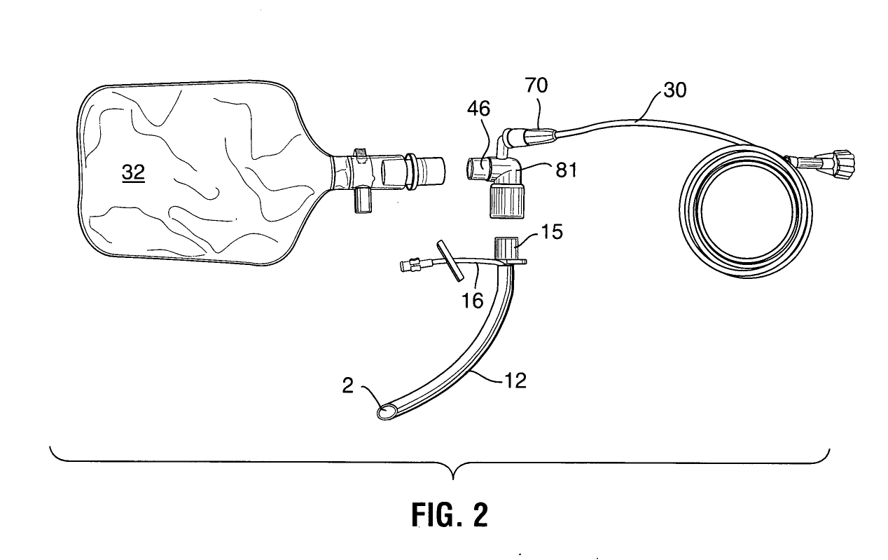

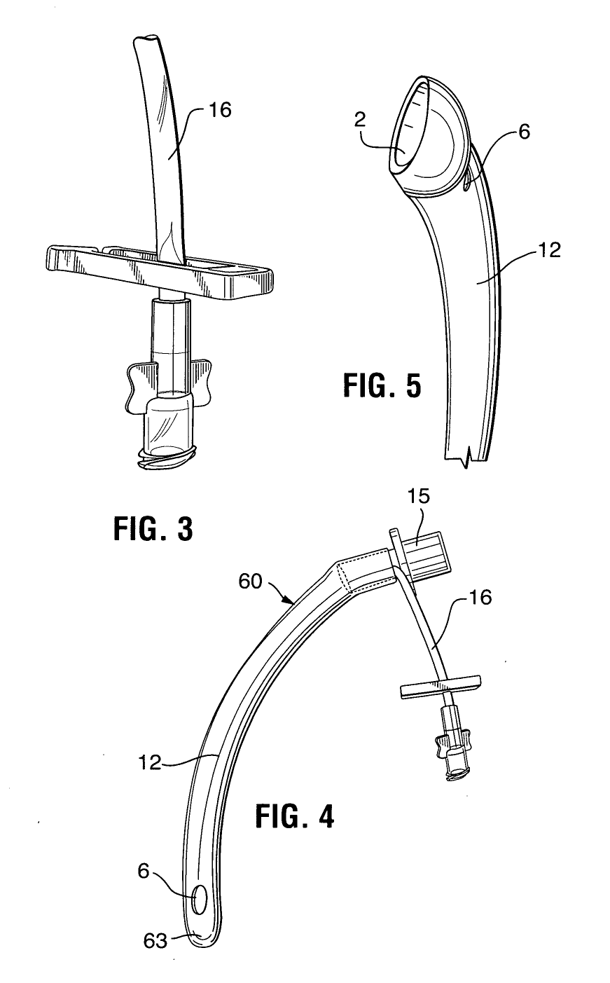

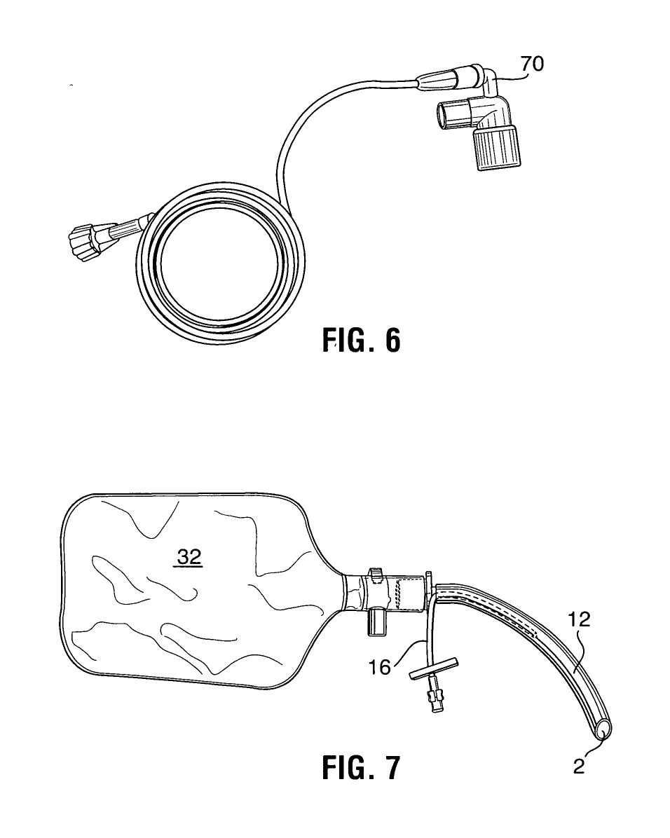

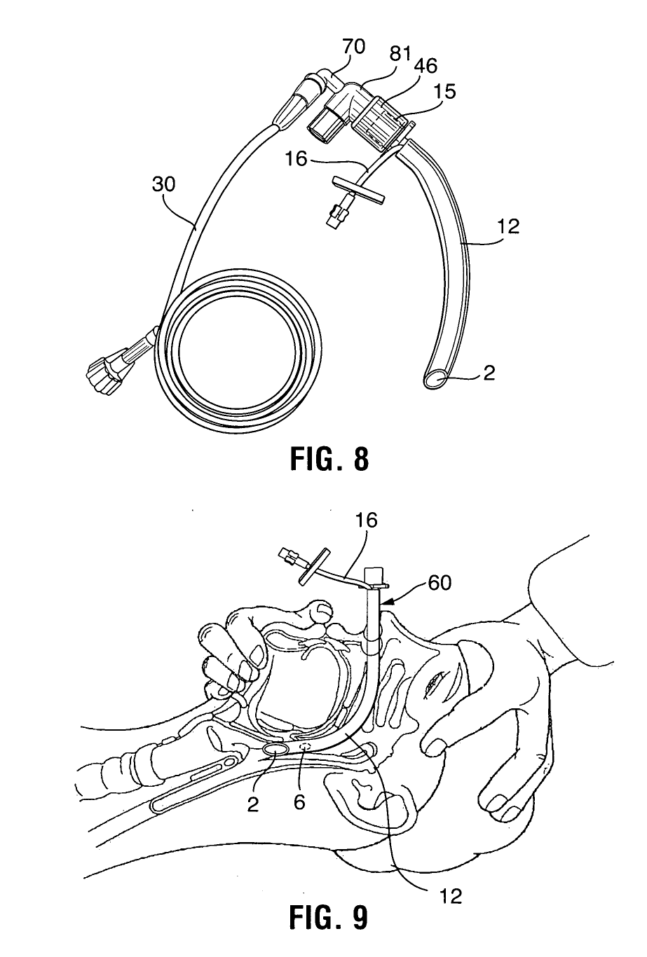

Referring now to the drawings and more particularly to FIGS. 1-10, thereof there is illustrated a respiratory ventilation device 8, which is constructed in accordance with one preferred embodiment of the present invention. The respiratory ventilation device 8 is utilized to ventilate a patient incapable of normal ventilation with current anesthetic techniques due to a risk for airway obstruction in surgical procedures requiring conscious sedation/monitored anesthesia care (MAC).

Considering now the respiratory ventilation device 8 in greater detail with reference to FIGS. 1-8, the respiratory ventilation device 8 generally comprises an air delivery assembly 61 and a single-lumen endopharyngeal nasal tube 60 with an eye shaped opening 6 at its distal end 63 and a cylindrical proximal end or male fitting part of the adaptor 15 of the endopharyngeal nasal tube 60. Contiguous to the distal end of the nasal tube 60 is an end-tidal CO2 monitoring port (ETCO2) 16. The distal end of the nasal tube 60 and proximal end of the end-tidal CO2 monitoring port 16 are enclosed in the proximal end of the flexible nasal ventilation tube 12. The flexible nasal ventilation tube 12 is made of a latex-free, non-toxic material.

On the distal end of the flexible nasal ventilation tube 12 is an eye opening 2 allowing air flow to the posterior pharynx. Contiguous to the distal eye opening 2 is an opening in the sidewall 6 of the flexible nasal ventilation tube 12 allowing for airflow to the posterior pharynx in the event that the distal eye is covered by body tissue. The opening in the sidewall 6 in cooperation with the distal eye opening 2 helps distribute positive pressure air into the airway of a patient and thus, helps prevent the tube 60 from being dislodged from the nostril of the patient due to excessive pressure buildup in the airway of the patient. In this manner the delivery of the positive pressure air into the airway of the patient causes the periglottic area within the airway to expand or distend and causes the larynx to open, thus facilitating the pulmonary exchange of air.

The air delivery assembly 61 generally includes adapters for cooperatively engaging and providing an air tight seal with a male fitting part of the adaptor 15 that is made to be quickly and easily wedged into the female fitting of multiple devices. The ventilation pump tube 30 is connected to the universal adaptor 81 by an air-tight, friction coupling between the nipple 70 which extends from the universal adaptor 81 and the proximal end of the ventilation pump tube 30. Once the ventilation pump tube 30 is joined with the universal adaptor 81, the universal adaptor can attach to the male fitting part of the adaptor 15. The modified JACKSON-REESE ambu bag 32 can either directly connect to the male fitting part of the adaptor 15 or connect to the universal adaptor's 81 male fitting part 47 and then be attached to the male fitting part of the adaptor 15.

As shown in FIG. 9, the method of using the respiratory ventilation device 8 involves placing the patient in a face up prone position with the head slightly back to make certain the airway of the patient is open. Neosynephrine nasal spray is used in the least obstructed nares and a tube size is selected by measuring from the tip of the patients nose to their earlobe or angle of jaw. The respiratory ventilation device 8 is then lubricated with a lubricating gel and inserted by its distal end 63 into the least obstructed nostril of a patient and pushed downward until completely inserted and the distal end 63 is positioned posterior to the pharynx. Next, necessary accessories for the procedure such as the modified Jackson-Reese ambu bag 32 or ventilation pump tube 30 can be attached to the endopharyngeal nasal tube 60 via the adaptors. When using the modified JACKSON-REESE ambu bag 32 for assisted ventilation, the medical practitioner squeezes the ambu bag 32 to discharge a wave of air into the nasal passageway and lungs of the patient. The mouth of the patient may be held shut during the squeezing action and then allowed to open during the exhaling of the air. The delivery of the positive pressure air causes the periglottic space to expand as well as opening the larynx to facilitate pulmonary exchange of air. The medical practitioner can then squeeze the modified JACKSON-REESE ambu bag 32 in synchronization with the inspiratory phase allowing sufficient time for exhalation by the patient.

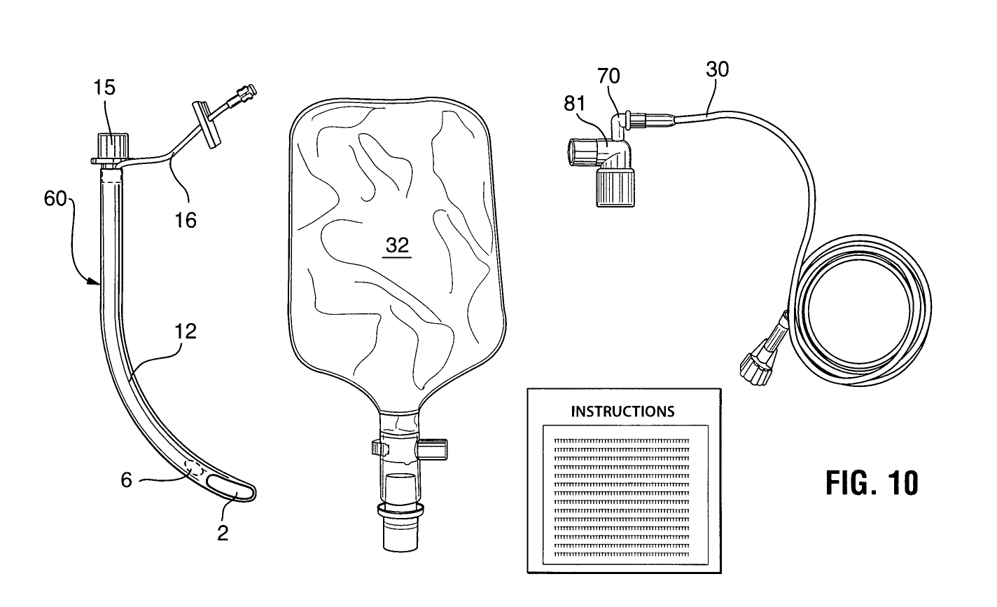

Referring now to the drawings and more particularly to FIG. 10, there is illustrated a ventilation respiratory ventilation kit, which is constructed in accordance with another preferred embodiment of the present invention. The ventilation respiratory ventilation kit is utilized to ventilate a medical patient. The respiratory ventilation kit generally includes a storage container that holds all the operative elements of the kit for easy in access and storage. In this regard, the container has a sufficient internal volume to hold the operative elements which include the endopharyngeal nasal tube, a modified JACKSON-REESE ambu bag for assisted ventilation, a ventilation pump tube, and a set of instructions. The kit contains a single-lumen endopharyngeal nasal tube with an eye shaped opening at its distal end and a cylindrical proximal end. Attached to the distal end is a flexible nasal ventilation tube made of a latex-free, non-toxic material. A small eye opening (commonly referred to as a Murphy eye) is disposed near the distal end and functions to allow the continued flow of air in the case that the distal end is blocked by body tissue. The ventilation pump tube is an elongated hollow tube having a female part threaded or friction fit first connector disposed at its distal end and a tight fitting sleeve second connector disposed at its proximal end. The first connector and the sleeve connector are both endotracheal tube connectors to permit one of them, to be quickly connected to the air bag and to permit the nasal tube end connector to be easily and quickly wedged into the proximal end of the nasal tube. Contiguous to the distal end of the nasal tube is an end-tidal CO2 monitoring port (ETCO2) 16.

The foregoing detailed description is given primarily for clearness of understanding and no unnecessary limitations are to be understood therefrom, for modification will become obvious to those skilled in the art upon reading this disclosure and may be made upon departing from the spirit of the invention and scope of the appended claims. Accordingly, this invention is not intended to be limited by the specific exemplification presented herein above. Rather, what is intended to be covered is within the spirit and scope of the appended claims.

I claim:

1. An endopharyngeal airway device for ventilating a patient incapable of normal ventilation with current anesthetic techniques due to a risk for airway obstruction in surgical procedures requiring conscious sedation monitored anesthesia care, comprising:
a single-lumen flexible endopharyngeal nasal ventilation tube including an eye shaped opening at its distal end;
said nasal tube including a adapter having a cylindrical proximal end removable coupling to a positive airway pressure device;
an end-tidal CO2 monitoring port contiguous to the distal end of said nasal tube;
an eye opening on the distal end of said endopharyngeal nasal ventilation tube allowing air flow to the posterior pharynx; and
said positive airway pressure device allows for controlled airflow to the patient during a procedure.

2. An endopharyngeal airway kit for ventilating a patient incapable of normal ventilation with current anesthetic techniques due to a risk for airway obstruction in surgical procedures requiring conscious sedation monitored anesthesia care, comprising:
a single-lumen endopharyngeal flexible ventilation nasal tube with an eye shaped opening at its distal end and a cylindrical proximal end adaptor for cooperatively engaging a fitting of an anesthetic application device comprising a positive airway pressure via Jackson-Reese modified ambu-bag;
an end-tidal CO2 monitoring port contiguous to the distal end of the nasal tube, said end-tidal CO2 monitoring port, wherein a distal end of the tube and proximal end of the end-tidal CO2 monitoring port are enclosed in the proximal end of said nasal tube;
said nasal tube including an eye opening on the distal end thereof allowing air flow to the posterior pharynx;
said nasal tube including an opening in the sidewall contiguous to the distal eye opening allowing for airflow to the posterior pharynx in the event that the distal eye is covered by body tissue; and
a modified Jackson-Reese ambu bag which allows for controlled airflow to the patient during a procedure

3. The method of using an endopharyngeal airway device for ventilating a patient incapable of normal ventilation with current anesthetic techniques due to a risk for airway obstruction in surgical procedures requiring conscious sedation monitored anesthesia care, comprising the steps of:
administering a neosynephrine nasal spray in the least obstructed nare;
selecting a flexible nasal ventilation tube size by measuring from the tip of the nose to the earlobe or angle of jaw;
applying lubricating gel to the endopharyngeal flexible nasal ventilation tube;
inserting the nasal tube into a selected nare with a gentle rotating motion, wherein when the nasal ventilation tube is in the correct position, it will extend from the distal end of the tube to the posterior of the pharynx;
connecting a cylindrical proximal end adaptor of said nasal tube to a fitting of an anesthetic application device comprising a positive airway pressure via Jackson-Reese modified ambu-bag; and
connecting an end-tidal CO2 monitoring port contiguous to the distal end of the nasal tube, said end-tidal CO2 monitoring port, wherein a distal end of the tube and proximal end of the end-tidal CO2 monitoring port are enclosed in the proximal end of said nasal tube to a CO2 monitoring device.
